(19) United States
(12) Patent Application Publication
(10) Pub. No.: US 2008/0147013 A1
(43) Pub. Date: Jun. 19, 2008

(54) Catheter shape-retaining cover

(76) Inventors: Thomas G. Breton, San Jose, CA (US); Neal H. Padilla, San Francisco, CA (US); Sheila V. Asuncion, San Jose, CA (US)

Correspondence Address: FULWIDER PATTON LLP, HOWARD HUGHES CENTER, 6060 CENTER DRIVE, TENTH FLOOR, LOS ANGELES, CA, 90045, US

(21) Appl. No.: 11/641,435

(22) Filed: Dec. 18, 2006

(51) Int. Cl. A61M 5/32

(52) U.S. Cl. 604/174

(57) ABSTRACT

The catheter shape-retaining cover includes an elongated tubular member having a predetermined non-linear shape and one or more inner lumens to receive a distal tip of a catheter. A tubular dispenser for the catheter may be attached to the elongated tubular member. A shaped mandrel may be placed in a first inner lumen of the sheath. The assembly may be heated to a temperature that makes the distal tip of the catheter become more plastic, and then cooled to pre-set the shape of the distal tip of the catheter. The distal tip of the catheter also may be hydrated in an inner lumen of the sheath, and the catheter may be hydrated in the tubular dispenser.

### BACKGROUND OF THE INVENTION

This invention relates generally to devices for interventional therapeutic treatment or vascular surgery for treatment of defects in the vasculature, and more particularly concerns a system and method for protecting and maintaining a desired shape of a non-linear segment of a catheter, such as a catheter for treatment of aneurysms. Although the catheter shape-retaining cover can be applied to any catheter that has a non-linear segment, it is most applicable for retaining the shape of the distal end of catheters.

Vascular interventional devices such as vaso-occlusive devices are commonly placed within the vasculature of the human body via an introducer catheter. Such vaso-occlusive devices can be produced in such a way that they will pass through a delivery lumen of an introducer catheter in a linear shape and take on a complex shape as originally formed after being deployed into the area of interest, such as an aneurysm. Such introducer catheters are typically quite flexible in order to be capable of negotiating twists and turns of the vasculature, and are often custom shaped at the time of use by the physician by means of a shaping wire and steam heating, such as to form a distal portion of the introducer catheter to a desired shape suited to a particular portion of the vasculature, for example. Recently, some manufacturers have produced catheters with a fixed predetermined shape to spare the physician the need to steam shape the catheter. To maintain the desired predetermined shape of catheters, the non-linear segments must be maintained in their pre-shaped configuration during packaging and storage. For example, the shape of catheters can change during sterilization or under moderate heat from shipping or storage. For this reason, pre-shaped segments are not suitable for the generally-linear packaging configuration of the hoop-style packaging common to straight introducer catheters and guidewires. Therefore, pre-shaped catheters have routinely been packed in cumbersome and more expensive thermo-formed trays.

One current standard technique for maintaining the desired shape is to place a wire or mandrel, also known as a stylet, formed into the desired shape, in the interior of the delivery lumen of the introducer catheter. The strength and rigidity of the shape retaining mandrel or stylet is limited by the inner diameter of the lumen of the introducing catheter, so that smaller introducer catheters required for delivering smaller vascular intervention devices in narrow, delicate passages of the vasculature can only accept thin, flimsy shape retaining mandrels, which can be inadequate for properly protecting and maintaining the desired shape of the distal tip of the introducer catheter. This approach also requires removal of the shape retaining mandrel from the delivery lumen of an introducer catheter prior to use of the catheter, and thereby increases handling of the catheter tip and a consequent potential for damage to the catheter. For example, even simply removing such a shaped mandrel from the lumen of an introducing catheter can result in damage to the delivery lumen of a catheter by dragging of the shaped mandrel through the delivery lumen of the catheter. In addition, plugging of the delivery lumen of an introducing catheter interferes with flushing of the catheter lumen, and may interfere with gas sterilization processing. Because the shaping mandrel must be removed prior to use, pre-shaped catheters are generally packaged in cumbersome trays, rather than in the convenient hoops in which straight catheters and guide wires are typically packaged. Current techniques for hydrating a catheter in a tray prior to use are messy, and involve unnecessary handling which can compromise the catheter's sterility and functionality.

Accordingly, it has been found that there is a need to maintain the desired shape of shaped segments of introducer catheters which accommodate hoop-style packaging, without requiring contact or opportunity for damage to the delivery lumen of an introducer catheter during removal of a shape retaining mandrel. There is also a need to allow the distal end of the catheter delivery lumen to be open for sterilization and flushing, and to allow for the use of common hoop-style packaging. The present invention meets these and other needs.

### SUMMARY OF THE INVENTION

The present invention accordingly provides for a catheter shape-retaining cover for protecting and maintaining a desired shape of a distal tip or any other non-linear segment of a catheter. The present invention also permits more options than present systems for the pre-shaping of catheter tips. For example, the assembly can be heated to a temperature which makes the catheter become more plastic, and when cooled, pre-sets the catheter according to the shape of the catheter shape-retaining cover, which thereafter protects the catheter from any tendency to retain a shape not in compliance with the shape of the catheter shape-retaining cover. Such a procedure allows for the formation of many predetermined shapes to accommodate the preferences of a variety of surgeons using such catheters.

Accordingly, in a presently preferred embodiment, the present invention provides for a catheter shape-retaining cover for protecting and maintaining a desired shape of a distal tip of a catheter, wherein the shape-retaining cover includes an elongated tubular member having a predetermined non-linear shape and having one or more inner lumens which removably receive a distal tip of a catheter. In a preferred aspect, the shape-retaining cover may include a tubular hoop dispenser for the catheter, with the catheter shape-retaining cover being attached to a distal end of the tubular hoop dispenser. In another presently preferred embodiment, the catheter shape-retaining cover may include a plurality of inner lumens.

In another presently preferred embodiment, the present invention provides for a catheter shape-retaining cover for protecting and maintaining a desired shape of a distal tip of a catheter, including an elongated tubular member having a first inner lumen and a second inner lumen, the second inner lumen operative to removably receive a distal tip of a catheter, and a mandrel disposed in the first inner lumen. In another aspect, the first inner lumen may have an internal diameter larger than an internal diameter of the second inner lumen. The mandrel is preferably formed in a shape corresponding to the desired shape of the distal tip of the catheter. In another presently preferred embodiment, the mandrel may have a generally rounded, concave cross-sectional shape.

The present invention accordingly also provides for a method for forming a shape in the distal tip of a catheter into a predetermined non-linear shape and maintaining the predetermined non-linear shape. A distal tip of a straight catheter is inserted into a packaging hoop containing a shape-retaining cover which has a desired shape corresponding to the predetermined non-linear shape of the desired catheter. The distal tip of the catheter and shape-retaining cover may then be heated to a temperature that makes the catheter become more plastic, and then cooled to pre-set the distal tip of the catheter according to the shape of the shape-retaining cover. The catheter tip is then maintained in the shape-retaining cover, and may be packaged in a tubular hoop dispenser and sterilized according to known industry methods. At the time of use, the catheter is removed from the hoop, which retracts the distal tip of the catheter from the shape-retaining cover.

In another presently preferred aspect, the distal tip of the catheter may be hydrated in an inner lumen of the shape-retaining cover. Where the shape-retaining cover includes a tubular dispenser for the catheter, with the shape-retaining cover attached to the distal end of the tubular dispenser, and the shape-retaining cover includes a first inner lumen and a second lumen receiving the distal tip of a catheter, the body of the catheter may be hydrated through at least one of the first inner lumen and the second inner lumen.

Other features and advantages of the present invention will become more apparent from the following detailed description of the preferred embodiments in conjunction with the accompanying drawings, which illustrate, by way of example, the operation of the invention.

### BRIEF DESCRIPTION OF THE DRAWINGS

FIG. 1 is a side elevational view of a first embodiment of the catheter shape-retaining cover with at least one inner lumen, placed over a distal portion of a catheter, and attached to a distal end of a tubular dispenser, according to the invention.

FIG. 2 is a side elevational view of the catheter shape-retaining cover and a distal portion of a catheter of FIG. 1, disassembled.

FIG. 3 is an end view of the distal end of the catheter shape-retaining cover of FIG. 2 according to the invention.

FIG. 4 is a cross-sectional view of the catheter shape-retaining cover taken along line 4-4 of FIG. 2.

FIG. 5 is a side elevational view of a second embodiment of the catheter shape-retaining cover assembled with a mandrel, and placed over a distal portion of a catheter, according to the invention.

FIG. 6 is a side elevational view of the catheter shape-retaining cover, mandrel and a distal portion of a catheter of FIG. 5, disassembled.

FIG. 7 is an end view of the distal end of the catheter shape-retaining cover of FIG. 6 according to the invention.

FIG. 8 is a cross-sectional view of the catheter shape-retaining cover taken along line 8-8 of FIG. 6.

### DETAILED DESCRIPTION OF THE PREFERRED EMBODIMENTS

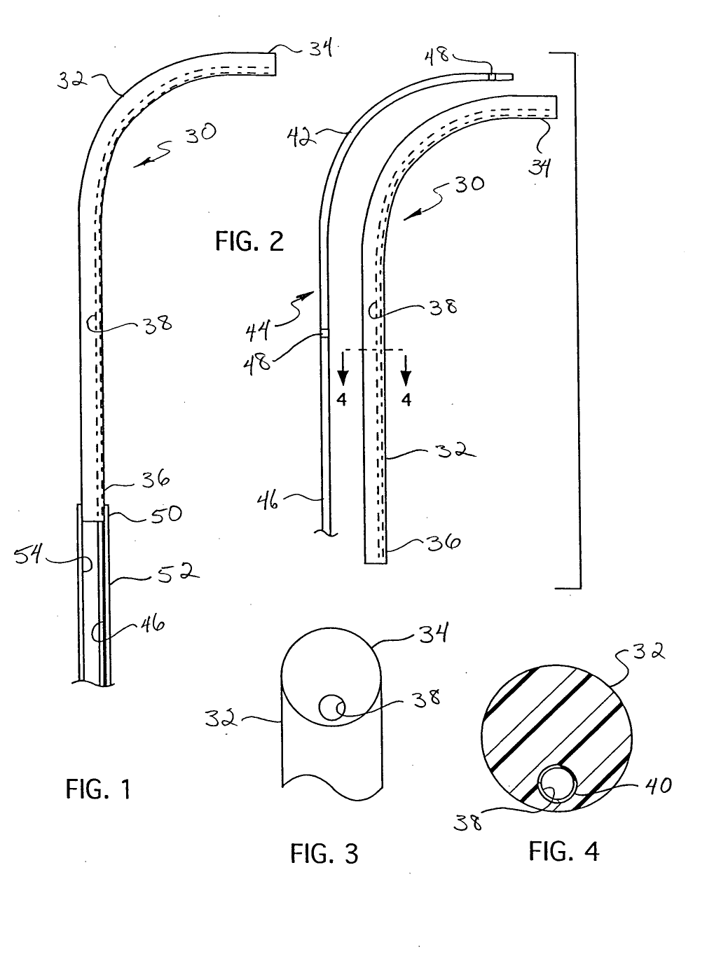

Referring to the drawings, which are provided for purposes of illustration and by way of example, in a first presently preferred embodiment, the present invention provides for a catheter shape-retaining cover 30 for protecting and maintaining a desired shape of a distal tip (or any other desired segment) of a catheter. The catheter shape-retaining cover includes an elongated tubular member 32, having a distal end 34 and a proximal end 36, and typically formed of a flexible plastic, such as a thermoplastic material, for example. One such thermoplastic material suitable for forming the body of the elongated tubular member is high density polyethylene, although other similar materials may also be suitable. As is illustrated in FIGS. 1-4, the elongated tubular member includes one or more inner lumens 38. The one or more inner lumens 38 may have an outer wall 40 formed of a material with a low coefficient of friction, such as polytetrafluoroethylene, for example, available commercially under the trade name “TEFLON,” although other similar materials with a low coefficient of friction, such as polyethylene or polypropylene, for example, may also be suitable. The one or more inner lumens removably receive a shaped distal tip 42 of a catheter 44, having a main body 46, and typically having one or more radiopaque markers 48. The diameter of the catheter and the distal tip of the catheter are typically sufficiently less than the diameter of the one or more inner lumens of the elongated tubular member of the catheter shape-retaining cover to allow the distal tip of the catheter to freely slide within the one or more inner lumens of the elongated tubular member. The catheter can then be stored, packaged, sterilized and flushed without affecting the desired shape of the distal tip of the catheter. The distal tip of the catheter thus may be removed from the one or more inner lumens by progressively sliding the distal tip of the catheter from the one or more inner lumens to unsheathe the distal tip of the catheter.

In another presently preferred aspect, the proximal end of the elongated tubular member of the catheter shape-retaining cover may be attached to a distal end 50 of a tubular dispenser 52 having an interior lumen 54 in which the body of the catheter may be contained to protect the catheter until it is time to be removed for use. The diameter of the body of the catheter is typically substantially less than the inner diameter of the interior lumen of the tubular dispenser to allow the catheter to freely slide within the interior lumen of the tubular dispenser, so that the catheter thus may be removed from the interior lumen of the tubular dispenser by progressively sliding the catheter from the interior lumen of the tubular dispenser and catheter shape-retaining cover to unsheathe the catheter from the tubular dispenser.

The distal tip of the catheter also may be hydrated in the one or more inner lumens of the elongated tubular member of the catheter shape-retaining cover by introduction of a hydrating fluid into the one or more inner lumens at the distal end of the elongated tubular member. When the elongated tubular member is attached to the distal end of the tubular dispenser, and the body of the catheter is disposed within the tubular dispenser, the body of the catheter may also be hydrated by introduction of hydrating fluid into the interior lumen of the tubular dispenser through one or more inner lumens.

The catheter shape-retaining cover also permits the shaping of the catheter within the shape-retaining cover to impart a pre-shaping of catheter tips or any other portion of the catheter. For example, a straight catheter may be inserted in the shape-retaining cover and then both are heated to a temperature which makes the catheter become more plastic, such as by exposing the catheter shape-retaining cover to a source of steam, and then allowing the catheter shape-retaining cover to cool. This pre-sets the shape of the distal tip of the catheter according to the shape of the catheter shape-retaining cover, and thereafter protects the distal tip of the catheter from any tendency to return to a shape not in compliance with the desired shape of the catheter shape-retaining cover. In such embodiments, the shape-retaining cover must be formed of materials that have higher melting points than the temperature used to shape the catheter. Such a procedure allows for the formation of a wide variety of pre-bends to accommodate the preferences of surgeons using such catheters. The catheter can then be stored, packaged, sterilized and flushed without affecting the desired shape of the distal tip of the catheter.

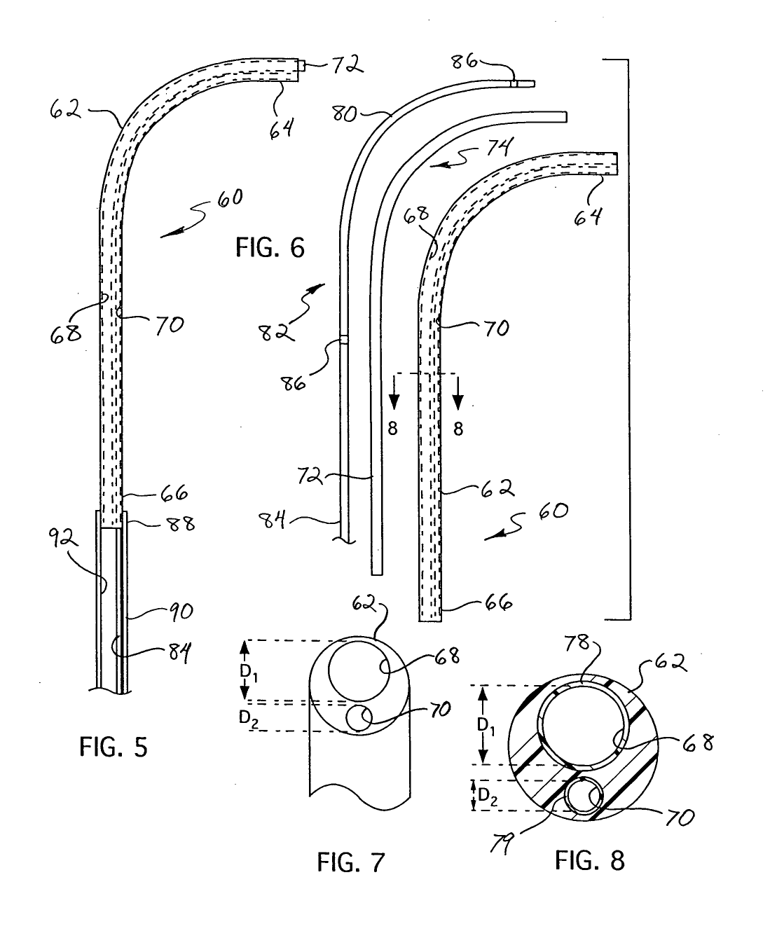

In a second presently preferred embodiment, illustrated in FIGS. 5-8 the present invention provides for a catheter shape-retaining cover 60 for protecting and maintaining a desired shape of a distal tip of a catheter. The catheter shape-retaining cover includes an elongated tubular member 62, having a distal end 64 and a proximal end 66, and typically formed of a flexible plastic, such as a thermoplastic material, for example. One such thermoplastic material suitable for forming the body of the elongated tubular member is high density polyethylene, although other similar materials may also be suitable. From an end view of the distal end of the catheter shape-retaining cover shown in FIG. 7, it can be seen that the elongated tubular member includes one or more first inner mandrel lumens 68, having an internal diameter C, and one or more second inner or catheter delivery lumens 70, having an internal diameter D₂, also shown in FIG. 8. In a presently preferred embodiment, the internal diameter of the one or more first inner lumens may be larger than the internal diameter of the one or more second inner lumens although this is not required as D₂ may be larger than D₁.

A shape retaining mandrel 72 having an end portion 74 having a shape corresponding to the desired shape of the distal tip of the catheter is received in the first inner lumen to maintain the shape of the elongated tubular member, and thus to maintain the desired shape of the distal tip of the catheter received in the shape-retaining cover. The catheter can then be stored, packaged, sterilized and flushed without affecting the desired shape of the distal tip of the catheter.

The mandrel is preferably formed of sufficient strength and rigidity to maintain the desired shape of the distal tip of the catheter. The mandrel is typically formed from a rigid or semi-rigid metal wire, such as stainless steel wire, for example, which can be formed in a shape corresponding to the desired shape of the distal tip of the catheter, although rigid or semi-rigid materials that may be shaped by a tool or by hand and that will retain a desired shape with sufficiently adequate strength may also be suitable, such as plastic, or other types of metals or alloys, for example.

Referring to FIG. 8, the one or more first inner or mandrel lumens typically have an outer wall 78, and the one or more second inner or catheter delivery lumens have an outer wall 79, that may be formed of a material with a low coefficient of friction, such as polytetrafluoroethylene, for example, available commercially under the trade name “TEFLON,” although other similar materials with a low coefficient of friction, such as polyethylene or polypropylene, for example, may also be suitable. The one or more second inner lumens removably receive a shaped distal tip 80 of a catheter 82, having a main body 84, and typically having one or more radiopaque markers 86. The diameter of the pre-shaped portion of the catheter is typically sufficiently less than the diameter of the one or more second inner lumens 70 of the catheter shape-retaining cover to allow the distal tip of the catheter to freely slide within the one or more second inner lumens of the catheter shape-retaining cover. The distal tip of the catheter thus may be removed from the one or more second inner lumens by progressively sliding the distal tip of the catheter from the one or more second inner lumens to unsheathe the distal tip of the catheter.

In another presently preferred aspect, the proximal end of the elongated tubular member of the catheter shape-retaining cover may be attached to a distal end 88 of a tubular dispenser 90 having an interior lumen 92 in which the body of the catheter may be contained to protect the catheter until it is time to be removed for use. The diameter of the body of the catheter is typically substantially less than the inner diameter of the interior lumen of the tubular dispenser to allow the catheter to freely slide within the interior lumen of the tubular dispenser, so that the catheter thus may be removed from the interior lumen of the tubular dispenser by progressively sliding the catheter from the interior lumen of the tubular dispenser to unsheathe the catheter from the tubular dispenser.

The distal tip of the catheter also may be hydrated in the one or more second inner lumens of the elongated tubular member of the catheter shape-retaining cover by introduction of a hydrating fluid into the one or more second inner lumens at the distal end of the elongated tubular member in which the catheter shape-retaining cover is situated. When the elongated tubular member is attached to the distal end of the tubular dispenser, and the body of the catheter is disposed within the tubular dispenser, the body of the catheter may also be hydrated by introduction of hydrating fluid into the interior lumen of the tubular dispenser through the one ore more second inner lumens.

The catheter shape-retaining cover advantageously can be provided with a predetermined non-linear shape, and can thus be used for protecting and maintaining a desired shape of the distal tip or any other portion of a catheter. The catheter shape-retaining cover also permits the shape of the catheter shape-retaining cover to impart a pre-bending of catheter tips according to the shape of the catheter shape-retaining cover, such as by heating the assembly to a temperature which makes the catheter become more plastic, such as by exposing the catheter shape-retaining cover to a source of steam. The catheter shape-retaining cover is then allowed to cool, to pre-set the shape of the distal tip of the catheter according to the shape of the mandrel in the catheter shape-retaining cover, and to thereafter protect the distal tip of the catheter from any tendency to return to a shape not in compliance with the desired shape of the catheter shape-retaining cover. Alternatively, the shape-setting process may be achieved during the sterilization process such as radiation, dry-heat, ethylene oxide or other sterilization process that may impart changes in the catheter's material which also will allow the catheter to conform to the shape of the shape-retaining cover, thereby eliminating the manufacturing set of setting the shape of the segment of the catheter. Such a procedure minimizes manufacturing steps while allowing for the formation of a wide variety of pre-bends to accommodate the preferences of surgeons using such catheters.

It should be appreciated that the shape-retaining cover may also be provided with one or more longitudinal slits to facilitate insertion or removal of the catheter from the shape-retaining cover, and that the shape-retaining cover may also be used to cover other portions of the catheter and is not limited to the distal tip only. In addition, the catheter and/or mandrel lumens need not be circular in cross-section as depicted in FIGS. 3, 4, 7 and 8. The apparatus of the invention is applicable to various types of catheters, and a wide variety of shaped or shapeable members can be used as a mandrel. In addition, the elongated tubing member may alternatively be formed from a wide variety of other materials that may also be suitable, such as polyurethane, nylons, silicone, polyetheretherketone (PEEK), polytetrafluoroethylene (PTFE), polyethylene terephthalate (PET), metals, ceramics and the like.

It will be apparent from the foregoing that, while particular forms of the invention have been illustrated and described, various modifications can be made without departing from the spirit and scope of the invention. Accordingly, it is not intended that the invention be limited, except as by the appended claims.

1. A catheter shape-retaining cover for protecting and maintaining a desired non-linear shape of a catheter, comprising:
an elongated tubular member having at least one inner lumen, said at least one inner lumen including an outer wall formed of a material with a low coefficient of friction, said at least one inner lumen being operative to removably receive a desired non-linear segment of a catheter, said elongated tubular member maintaining the predetermined non-linear shape of said desired non-linear segment of the catheter.

2. The catheter shape-retaining cover of claim 1, further comprising a tubular dispenser for the catheter, said elongated tubular member being attached to the tubular dispenser at some point along the length of said tubular dispenser.

3. The catheter shape-retaining cover of claim 1, wherein said elongated tubular member comprises a plurality of inner lumens.

4. The catheter shape-retaining cover of claim 3, wherein at least one of the plurality of the lumens contains a mandrel configured to maintain the non-linear shape of the elongated tubular member.

5. A catheter shape-retaining cover in combination with a catheter for protecting and maintaining a desired shape of a segment of the catheter, comprising:
an elongated tubular member having at least one inner lumen, said at least one inner lumen including an outer wall formed of a material with a low coefficient of friction, said elongated tubular member having a predetermined non-linear shape; and
a catheter having a catheter segment with a desired shape consistent with said predetermined non-linear shape, said catheter segment being removably received within said at least one inner lumen.

6. The catheter shape-retaining cover of claim 5, further comprising an elongated tubular dispenser for said catheter, said elongated tubular member being attached to the tubular dispenser at some point along the length of the tubular dispenser corresponding to the location of the desired shape of the segment of the catheter.

7. The catheter shape-retaining cover of claim 5, wherein said elongated tubular member comprises a plurality of inner lumens.

8. The catheter shape-retaining cover of claim 7, wherein at least one of the plurality of lumens contains a mandrel configured to maintain the non-linear shape of the elongated tubular member.

9. A catheter shape-retaining cover for protecting and maintaining a desired shape of a segment of a catheter, comprising:
an elongated tubular member having a first inner lumen and a second inner lumen, said second inner lumen configured to removably receive a segment of a catheter; and
a mandrel disposed in said first inner lumen.

10. The catheter shape-retaining cover of claim 9, wherein said first inner lumen has an internal diameter larger than or equal to an internal diameter of said second inner lumen.

11. The catheter shape-retaining cover of claim 9, wherein said first inner lumen has an internal diameter smaller than or equal to an internal diameter of said second inner lumen.

12. The catheter shape-retaining cover of claim 9, wherein said second inner lumen has an outer wall formed of a lubricious polymer.

13. The catheter shape-retaining cover of claim 9, wherein said mandrel is formed from wire.

14. The catheter shape-retaining cover of claim 13, wherein said mandrel is formed in a shape corresponding to said desired shape of said segment of said catheter.

15. A catheter shape-retaining cover in combination with a catheter for protecting and maintaining a desired shape of a segment of the catheter, comprising:
an elongated tubular member having at least a first inner lumen and a second inner lumen;
a catheter having a segment with a desired shape, said catheter segment being removably received in said second inner lumen; and
a mandrel disposed in said first inner lumen.

16. The combination of claim 15, wherein said first inner lumen has an internal diameter larger or equal to an internal diameter of said second inner lumen.

17. The combination of claim 15, wherein said mandrel is formed from wire.

18. The combination of claim 15, wherein said mandrel is formed in a shape corresponding to said desired shape of said distal tip of said catheter.

19-25. (canceled)

27. (canceled)
